(19) United States
(12) Patent Application Publication
(10) Pub. No.: US 2014/0214010 A1
(43) Pub. Date: Jul. 31, 2014

(54) DRUG DELIVERY DEVICE WITH COMPRESSIBLE FLUID CHAMBERS

(71) Applicants: Youti Kuo, Penfield, NY (US); Michael Violante, Pittsford, NY (US)

(72) Inventors: Youti Kuo, Penfield, NY (US); Michael Violante, Pittsford, NY (US)

(21) Appl. No.: 14/111,026

(22) PCT Filed: Apr. 18, 2012
(86) PCT No.: PCT/US12/33984

§ 371 Date: Apr. 2, 2014

Related U.S. Application Data
(60) Provisional application No. 61/517,231, filed on Apr. 18, 2011.

(51) Int. Cl. A61M 5/142

(52) U.S. Cl. CPC A61M 5/14276
USPC 604/891.1

(57) ABSTRACT

A drug delivery device utilizing compressible fluid chambers for actuation is disclosed. The device includes a compressible dispensing chamber situated in a first compartment and a compressible filler-fluid chamber in flow communication with the first compartment. The compressible dispensing chamber is attached with a reciprocating plunger for dispensing the drug fluid. The drug chamber is refillable with a refill container that uses a compressible pouch containing the refill drug fluid. The refilling process is failsafe enabled by the insertion of the refill container needle only at the correct position to activate an internal contact switch. The compressible-wall configurations enable low dispensing forces without leakage of the drug fluid.

### FIELD OF THE INVENTION

This invention relates to a drug delivery device using compressible fluid chambers for drug delivery and refill.

### BACKGROUND

Drug delivery by means of injections, inhalation, transdermal or swallowing pills or capsules generally result in varying drug concentrations between dosings. Many diseases would be better treated if the therapeutic drug were given so as to obtain a more or less constant drug level in the region of interest, especially if systemic drug concentrations could be maintained at or near zero thereby minimizing side effects. Implantable drug delivery devices attempt to achieve this by delivering small amounts of drug to a specific body cavity on a frequent basis. These delivery systems also are capable of protecting drugs which are unstable in vivo and that would normally require frequent dosing intervals. Implantable drug delivery devices include polymeric implants, implantable osmotic pump systems, and micro-pumps.

Polymeric implants, used extensively in controlled drug delivery systems, include non-degradable polymeric reservoirs and matrices, and biodegradable polymeric devices. In both cases the drug is released by dissolution into the polymer and then diffusion through the walls of the polymeric device. The release kinetics of drugs from such systems depend on both the solubility and diffusion coefficient of the drug in the polymer, the drug load, and, in the case of the biodegradable systems, the in vivo degradation rate of the polymer. Examples of polymeric implants include simple cylindrical reservoirs of medication surrounded by a polymeric membrane and homogeneous dispersions of drug particles throughout a solid matrix of non-degradable polymers. Biodegradable polymeric devices are formed by physically entrapping drug molecules into matrices or microspheres. These polymers dissolve when implanted or injected and release drugs.

Another method for controlled prolonged delivery of a drug is the use of an implantable osmotic pump. An osmotic pump is generally in a capsule form having permeable walls that allow the passing of water into the interior of the capsule containing a drug agent. The absorption of water by the water-attracting drug composition within the capsule reservoir creates an osmotic pressure within the capsule to push the drug out of the capsule to the treatment site.

Implantable micro-pumps for drug delivery applications usually include a permeable membrane for controlled diffusion of a drug into the body from a suitable reservoir. Such devices are limited in application primarily since the rate at which the drug is delivered to the body is completely dependent upon the rate of diffusion through the permeable membrane. With these devices the rate of drug delivery to the body may be affected by differing conditions within the body. In addition, such systems make no provision for the adjustment of the rate or time interval for drug delivery, nor can the delivery rate be easily varied.

Although polymeric implants, osmotic pumps and micro-pumps may provide a relatively steady rate of drug release, some drugs are more effective given in intervals. Implantable infusion pumps can be programmed to deliver drugs at very precise dosages and delivery rates. These pumps may have a feedback device that controls drug delivery according to need. With the current development of electronics and miniaturization of pumps and sensors, various vital signs can be monitored leading to feedback systems such as for monitoring blood glucose levels and delivering insulin when needed. The size of the pump depends on the amount of drug and the intended length of treatment. A barrier in feedback technology in using an implantable sensor is the problem of body proteins causing reduced sensitivity of the sensors, compromising the reliability of the sensor input.

### PRIOR ART

The following provides prior art related to features of drug delivery devices.

U.S. Pat. No. 7,377,907 by Shekalim provides an insulin pump that supplies insulin in a pre-pressurized chamber through a flow control valve. Precise metering is achieved by a piezoelectric actuator. The insulin in the chamber is pressurized and dispensed by a piston, which is driven by a biased spring. The device also includes a pressure regulator, a removable cartridge unit containing a pre-pressurized fluid reservoir, and an electronic package for the programming of basal rates. Nevertheless, patients with a portal device are at risk for trans-cutaneous infections.

On the use of two fluidic drug chambers, U.S. Pat. No. 5,607,418 by Arzbaecher provides an implantable drug delivery device having a deformable dispensing chamber within a deformable reservoir chamber. In this configuration, the dispensing flow rate of the dispensing chamber is designed to be greater than the refilling flow rate from the reservoir chamber and that the reservoir chamber automatically refills the dispensing chamber following discharge of a dispensing portion of the fluidic drug. Because the dispensing rate is greater than the refilling rate across the internal valve between the two deformable chambers, a partial vacuum may be created in the two chambers resulting in unstable dispensing rate or interruption of the dispensing flow to the treatment site. The deformable dispensing chamber within a deformable reservoir chamber cannot ensure the drug flow rate in and out of the dispensing chamber and the reservoir chamber are equal.

On refilling, U.S. Pat. No. 7,347,854 by Shelton, et al. relates to a process of refilling an implantable drug delivery device. The controller in accordance with this invention is programmed to determine the volume of the old drug remaining in the reservoir. The controller then monitors the subsequent delivery of the old drug to the patient to determine when the remaining old drug has been cleared from the device. Accordingly, the controller adopts a new dispensing profile for the drug refilled into the reservoir. The process as described in this patent is limited to the general practice of adding new drug after using up the original drug in the reservoir. No specific refill steps such as retracting a piston, closing a dispensing tip and using a passive syringe are addressed. In fact, a programmable pump allows changing the dispensing profile at any time depending on the need of a patient prior to using up the existing drug in the reservoir.

An implantable drug delivery pump of U.S. Pat. No. 6,283,949 by Roorda discloses a method of dispensing drug at a controllable rate from a reservoir. The pump includes a reservoir, a dispensing chamber, a compressible dispensing tube attached to the dispensing chamber, and a rotating-arm actuator for applying a compressive force onto the dispensing tube to deliver the drug through a catheter. The rotating-arm actuator allows additional drug drawn into the dispensing tube from the reservoir, which can be refilled. A one-way intake valve is used and the reservoir can be refilled through a septum. In this method, rotational actuator compressive force is moved in a direction from the intake end towards an outlet end and the reservoir is limited to a circular configuration to accommodate the rotating arm. Because the rotational compressive force is one-directional, its refilling of the reservoir chamber must use active-plunger type of refill syringe, which allows for injection of refill drug even its needle being inserted into wrong locations.

On drive means for imparting a piston or plunger motion a pump device, a piezoelectric motor driven by electric pulses can be used. U.S. Pat. No. 6,940,209 by Henderson provides a piezoelectric lead screw motor for driving an assembly that contains a threaded shaft and a threaded nut. Subjecting the threaded nut to piezoelectric vibrations causes the threaded shaft to simultaneously rotate and translate in the axial direction. A drive product based on the concept called Squiggle motor is commercially available. The SQUIGGLE SQ-306 model is 10 mm in length and 4 mm in diameter, and achieves precision levels in the micron range. The motor's power efficiency enables long battery life, which is a critical factor for implanted medical devices. Its motor driver board including ASIC, resonant inductors, Boost circuit and FWD diode can be packaged into 10 mm×10 mm×1.5 mm size.

### SUMMARY OF INVENTION

An implantable drug delivery device of the present invention includes a compressible dispensing chamber situated in a first compartment and a compressible filler fluid chamber which is in flow communication with the first compartment. The compressible dispensing chamber is attached with a reciprocating plunger for dispensing drug doses and the compressible walls of the reservoir chamber are contracted in responding to the dispensing actions of the plunger. The filler fluid chamber contains an inert fluid to fill the spaces in the first and the second compartments evacuated by the movements of the plunger and the compressible reservoir to prevent forming a partial vacuum in the compartments to ensure reliable performance of the pumping actions.

One embodiment of this invention comprises an implantable drug delivery device with a compressible dispensing chamber situated in a first compartment, a compressible reservoir drug chamber situated in a second compartment, and a compressible filler fluid chamber which is in flow communication with the first and the second compartments. The compressible dispensing chamber is attached with a reciprocating plunger for dispensing drug doses and the compressible walls of the reservoir chamber are contracted in responding to the dispensing actions of the plunger. The filler fluid chamber contains an inert fluid to fill the spaces in the first and the second compartments evacuated by the movements of the plunger and the compressible reservoir to prevent forming a partial vacuum in the compartments to ensure reliable performance of the pumping actions. The reservoir chamber can be refilled by a refill container, which uses a compressible chamber or collapsible pouch to contain the drug fluid. The refilling process is activated by the insertion of the needle of the refill container into the septum of the drug delivery device to block the flow path to the catheter and trigger an internal contact switch to activate the movement of the plunger. The backward movement of the plunger draws in the drug fluid from the refill container into the reservoir drug chamber. The automatic needle-activation refilling process requires precise insertion location to ensure failsafe refilling of the drug delivery device.

Key components of the dual chamber implantable pump device comprise: 1.) a compressible dispensing drug chamber situated in a first compartment, 2) a compressible reservoir drug chamber situation in a second compartment, 3) a one-way valve placed between the dispensing and the reservoir chambers, 4) a plunger attached to the dispensing drug chamber, 5) drug fluid contained in the dispensing and the reservoir drug chambers, 6) a compressible filler fluid chamber containing an inert filler fluid in flow communication with the spaces in the first and the second compartments unoccupied by the drug fluid, 7) a catheter with a flow channel and a contact switch functioning as a valve for blocking the flow channel upon the insertion of a refill container needle to activate the pumping motion of the plunger, 8) a control board containing a motor driver, a microprocessor and a battery, 9) a software program controlling the movement of the plunger.

### DESCRIPTION OF THE DRAWINGS

FIG. 1a is a front cross-section view of an implantable drug delivery device having compressible fluid chambers.

FIG. 1b is a top cross-section view of FIG. 1a showing cross-section areas of the dispensing and the reservoir chambers and a flow path between the two chambers.

FIG. 1c is a side cross-section view of the implantable drug delivery device of FIG. 1a showing a one-way check valve.

FIG. 2a is a compressible dispensing chamber of bellows configuration with its base attached with a plunger.

FIG. 2b is a front cross-section view of the plunger of FIG. 2a showing an internal drive coupling.

FIG. 3a shows the implantable drug delivery device of FIG. 1a with both dispensing chamber and reservoir chamber compressed to their most compressed positions.

FIG. 3b shows the implantable drug delivery pump device of FIG. 3a with a refill container needle inserted and pressing against a contact switch.

FIG. 4a is a front cross-section view of an implantable drug delivery device showing soft-layer filler-fluid chamber wrapped around two side walls and the bottom wall of the device housing.

FIG. 4b is a side cross-section view of the implantable drug delivery device of FIG. 4a.

FIG. 4c is a C-C cross-section of FIG. 4a showing flow gaps on housing walls connecting the filler fluid chamber with the first and second compartments for the filler fluid.

### DETAILED DESCRIPTION OF THE INVENTION

In the following descriptions, implantable drug delivery device, drug delivery device and infusion pump are used interchangeably.

To maintain a more constant rate of dispensing drug dosages, it is desirable to have an implant pump capable of precisely delivering a small amount of drug volume in the nano-liter range at each step of piston movement. It is desirable to infuse such minute dosages at time intervals appropriate for sustaining drug efficacy while avoiding side effects. And it is desirable to have an automated refilling process to prevent the injection of drug outside the implant pump into body tissues while refilling the pump.

With the above-mentioned prior art limitations of implantable drug delivery device technology, an objective of the present invention is to provide a divided drug chamber to enable small reciprocating motions of piston for dispensing small precise amounts of drug volume at each step of the piston forward movement. Another objective is to infuse such drug volumes at desirable time intervals to sustain drug efficacy while minimizing side effects. And still another objective is to have an automated refilling process to prevent injecting the drug outside the implant pump into body tissues during the refilling process. Additionally another objective is to provide automatic notifications for drug refilling and battery low status with an internal signal to alert the patient to take corrective action.

### Device Configuration

An implantable drug delivery device or infusion pump of the present invention includes a compressible dispensing chamber situated in a first compartment, a compressible reservoir drug chamber situated in a second compartment, and a compressible filler fluid chamber which is in flow communication with the first and the second compartments.

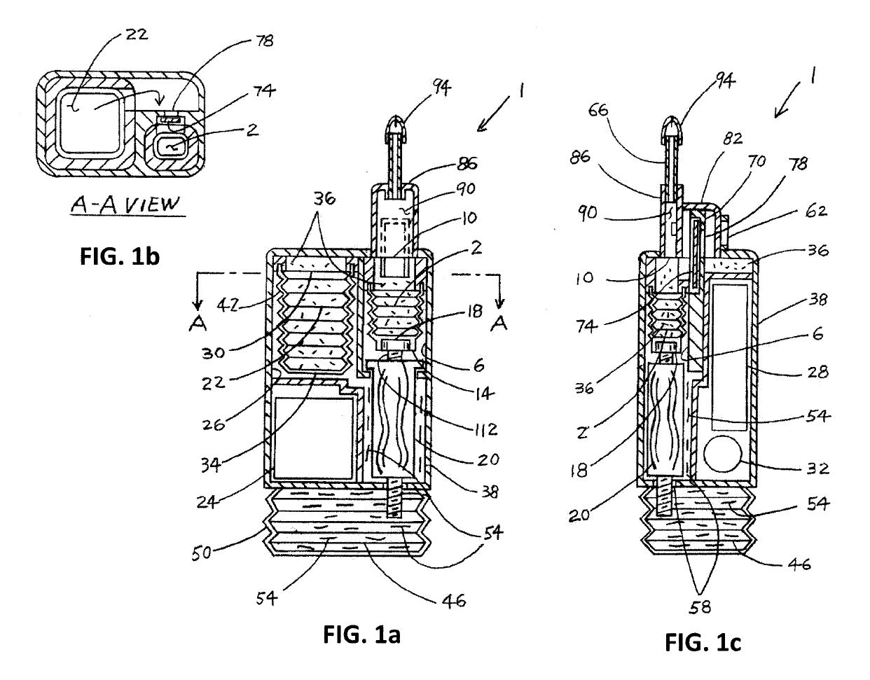

FIG. 1a, 1b and 1c show an implantable drug delivery device 1 of the present invention. As shown in FIG. 1a, a compressible dispensing chamber 2 situated inside first compartment 6 has an open end with first outlet 10 and a closed end with first base 14. The first base is attached with a plunger 18, which is an actuator for dispensing drug doses. The compressible reservoir chamber 22 situated inside second compartment 26 has an open end with second outlet 30 and a closed end with second base 34. Both first and second compartments are supported by housing walls 38. The compressible walls 42 of the reservoir chamber 22 contract or expand in responding to the forward and backward movements of the plunger 18. A compressible filler fluid chamber 46 supported by the housing walls 38 has collapsible walls 50, as shown in bellows configuration, containing a filler fluid 54 in communication with first compartment through flow path 58 (shown in FIG. 1b) and with second compartment with flow path 60. A filler fluid 54 is an inert fluid biocompatible with the drug fluid and body tissues. The collapsible walls 46 contracts as the filler fluid moves into first and second compartments in the space unoccupied by the dispensing and reservoir chambers in response to the reduced volume of the drug fluid as the plunger moves forward to dispense the drug fluid out of the first outlet. The filler fluid chamber contains an inert fluid to fill the spaces evacuated by the movements of the plunger and of the compressible reservoir to prevent forming a partial vacuum in the first and the second compartments to ensure reliable performance of the pumping actions. The compressibility of the dispensing chamber 2, of the reservoir chamber 22 and of the filler fluid chamber 46 are represented by the bellows configuration. Alternatively, the compressibility can be achieved by using collapsible walls of a thin film flexible pouch, whose walls can be folded to reduce its internal volume. The reservoir chamber, which will be described later, can be refilled by a refill container, using a collapsible pouch filled with the drug fluid. The refilling process will be described in later section.

The flow path between the dispensing chamber 2 and reservoir chamber 22 is illustrated in FIG. 1b, which is a top cross-section view of the device 1 shown in FIG. 1a. FIG. 1b shows the division between dispensing chamber 2 and reservoir chamber 22 by wall 70 and the one-way valve 74. The one-way valve overlaps flow path opening 78 (also shown in FIG. 1c). FIG. 1c shows the extension of the dividing wall 70 and the one-way valve 74 into septum 62 of the implant device 1. The one-way valve provides the flow path 78 between the dispensing chamber 2 and the reservoir chamber 22. The one-way valve closes when the plunger moves toward the first outlet and the one-way valve opens when the plunger moves away from the first outlet causing the drug fluid to flow from the reservoir chamber 22 into the dispensing chamber 2. The plunger is driven by a drive means for infusing the drug fluid through first outlet 10 and reducing the volume of the drug fluid in the dispensing chamber 2. In this divided drug chamber configuration, the cross-section flow area of the dispensing chamber may be much smaller than the cross-section flow area of the reservoir chamber such that the drug dosage dispensed at each step of the plunger forward movement can be very small independent of the size of the reservoir chamber.

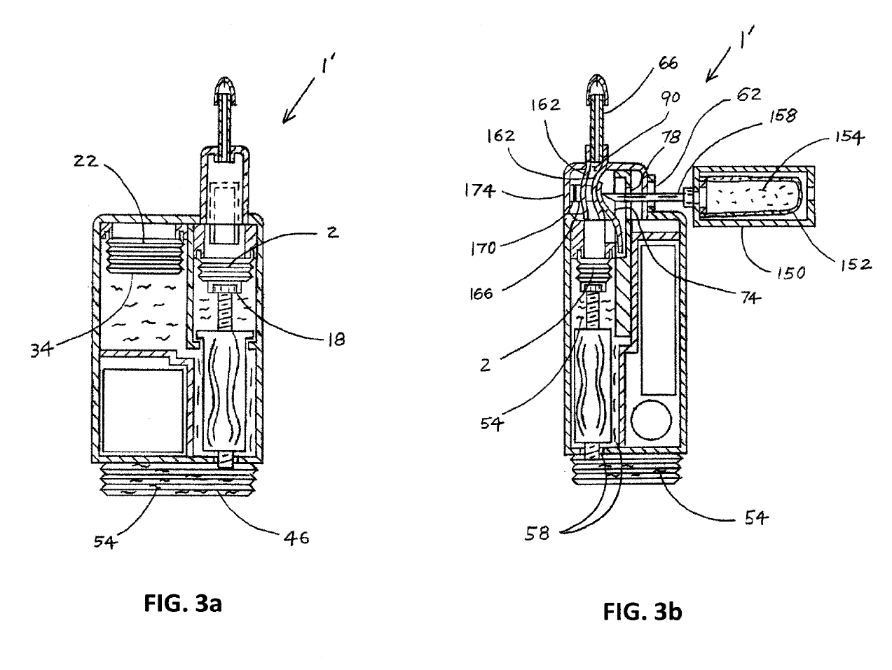

Additionally, catheter 66 is attached to first outlet 10 of dispensing drug chamber 2 in communication with the drug fluid 36. The septum wall 82 includes septum 62 for inserting needle of a refill container to fill the drug chambers (to be described later in FIG. 3b). Septum wall 82 also includes collapsible catheter base walls 86 forming an outlet flow channel 90. A dispensing cap in a form of a slit-valve 94 is attached at the dispensing end of the catheter 66. A normally-closed slit-valve prevents backflow of fluids from the external environment into the device. The slit-valve is forced to open by the forward movement of the plunger exerting pumping pressure allowing the drug to be dosed from the dispensing chamber to the treatment site. When implanted into a patient's body, the catheter 66 can be placed to a treatment location where the drug is dispensed.

### Bellows Configuration

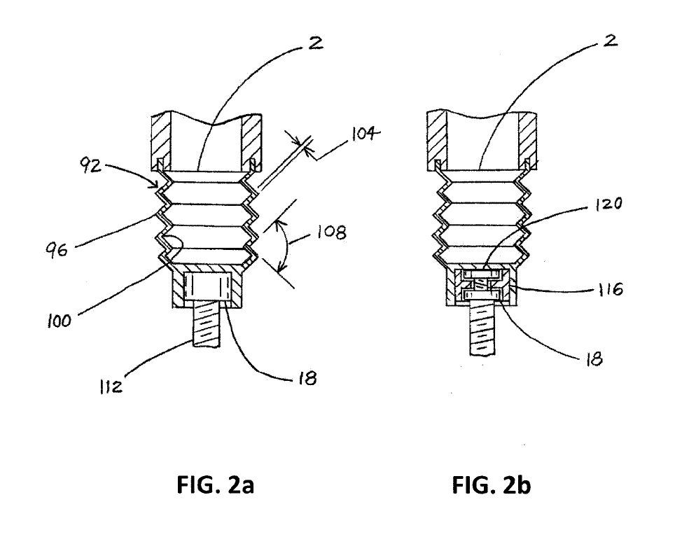

An embodiment of a compressible chamber of the present invention is of bellows configuration as shown in FIGS. 2a and 2b. A bellows used as a compressible chamber consists of a number of ring walls 92 forming a series of ridge folds 96 and valley folds 100. Preferably a bellows is molded with a biomedical compatible elastomer such as silicone rubber. A geometry of a bellows is characterized by the number of ring walls, the wall thickness 104 and the fold angle 108, the cross-sectional shape, the maximum and the minimum diameters (for circular cross-section) and the length of the bellows. A bellows is designed for resiliency and uniform compressibility along its length. A bellows may have a circular or non-circular cross-sectional shape. The dispensing bellows 2 shown in FIGS. 1a, 1b and 1c has non-circular cross-sectional shape as shown in FIG. 1b. The bellows expands with the fold angle increased under extension force and contracts with the fold angle decreased under a compression force. When a bellows is full of fluid its compression can expel a constant volume of the fluid at each constant increment of compression. The wall thickness of the bellows is designed for resiliency, durability and for desirable spring stiffness, which defines force and displacement relationship. To function as a drug chamber in the present invention, a bellows is mounted inside an compartment whose inside diameter is larger than the outside diameter of the bellows to form a spatial clearance to prevent contact between the bellows walls and the compartment wall. Therefore, there is no sliding friction between the bellow walls and compartment walls. The clearance allows the space to be filled with the filler fluid to result in hydraulic pressure that hinders any lateral movement of the bellows. In comparison with other compressible chamber configuration such as a collapsible pouch, the bellows configuration has advantage in its well controlled deformation under axial force.

### Reciprocating Plunger Motion

In the present invention a plunger is an actuator to perform a reciprocating motion. A plunger can be driven by a solenoid, a linear motor, a stepper motor, or by a magnetic force. In one embodiment motor 20 and motor driver 24 are used as shown in FIG. 1a. The motor driver, which is programmable, is under the control of a microprocessor 28. The motor, the motor driver and the microprocessor are all powered by the battery 32. FIGS. 2a and 2b also shows a means of attaching plunger 18 to dispensing chamber 2 with FIG. 2b describing an internal drive coupling. The drive coupling converts the rotational motion of threaded rod 112 to a linear motion of the plunger using a non-rotational sleeve 116 press-fit into the first base 14, and a free-to-rotate retainer 120. The plunger is designed not to contact compartment wall to cause sliding friction.

The drug fluid is pushed by the forward movement of the plunger. During the forward motion of the plunger the one-way valve 74 is forced to close and the cap in the form of slit-valve at the end of the catheter is forced to open to dispense the drug fluid. During the backward motion of the plunger a partial vacuum is created in the dispensing chamber that causes the slit-valve to close and the one-way valve to open. As a result, the drug fluid 36 from the reservoir chamber 22 enters the dispensing chamber 2 through the valve opening 78. Simultaneously, the reservoir chamber contracts with the second base 34 moving forward, which induces the filler fluid 54 to fill the space left by the contraction of the reservoir chamber through the flow gaps 60 and to fill the space left by the movement of the plunger. In repeated reciprocating motion of the plunger, the drug fluid is incrementally dispensed and the second base 34 of the reservoir chamber is moving forward in each cycle. This process continues until the reservoir drug chamber is depleted or empty. In the empty state, the space behind the second base is full of the filler fluid.

### Priming Steps

To avoid dead spaces, voids or air pockets in a drug delivery device of the present invention, the priming steps for complete filling of the device with drug fluid and filler fluid are as follows. To start with a new and empty condition, 1) squeeze and keep the slit valve at open condition, then activate the motor to move the plunger to the upper travel limit, 2) fill the filler fluid chamber with inert fluid until both the first and the second compartments full of the inert fluid with the reservoir chamber compressed to its upper limit position, 3) insert a pre-filled passive refill container (to be described in later section) with the drug fluid to the tip of needle into septum without opening one-way valve, 4) withdraw the filler fluid with a syringe until the reservoir chamber being filled up with the drug fluid completely, 5) with the slit valve remained open, push the refill container needle further to open the one-way valve, 6) withdraw the plunger to fill up the septum, the top space in the dispensing chamber and the catheter and to expel the air through the slit valve, 7) release the slit valve to resume its self-closing position and retract the plunger all the way to the lower travel limit to draw the drug fluid to fill the dispensing chamber completely, 8) remove the refill container from the septum, 9) if necessary, adjust the fluid pressure in the three fluid chambers by withdrawing or injection (filler) inert fluid into the filler fluid chamber.

Refilling Process

After an implantable drug delivery device of the present invention is depleted of drug fluid after repeated pumping, both the dispensing chamber 2 and the reservoir chambers 22 are at their most contracted conditions with the plunger 18 and the second base 34 at their upper limit positions as shown in FIG. 3a. Refilling of the dispensing and reservoir drug chambers can be accomplished by inserting a refill container 150 into the septum 62 of the infusion pump device 1 as shown in FIGS. 3a and 3b. Refill container 150 with collapsible pouch 152 containing refill drug 154 is inserted into the septum 62 of the device 1. Correct positioning of the refill container enables the needle 158 push open the one-way valve 74 toward the catheter base walls 162, which are deflectable and collapsible. Pushing of the needle 158 further causes the catheter base wall 162 contact each other to block the flow channel 90 of the catheter 66, at which position an injection hole (not shown) on the needle wall is in flow communication with both the dispensing and the reservoir chambers. Preferably the refill container has an internal one-way valve (not shown) to prevent backflow from the injection opening into the drug pouch 152 although any backflow is hindered by the small injection opening and the needle's long narrow flow channel. Forcing the catheter base walls 162 to touch each other also enables the activation of an electrical switch position behind the base walls. The electrical switch can be a Hall-effect switch or a contact switch formed by two thin electrode elements. The electrical switch is in electrical communication with the motor driver to activate the reciprocating or pumping motion of the plunger. The use of Hall-effect switch is known in the art. One embodiment of the electrical switch is a contact switch, in which first electrode 166 is on the backside of catheter base wall and second electrode 170 is on an opposing rigid wall 174. Both electrode plates are not in contact with drug fluid. As a reverse of the dispensing function, retraction or backward movement of the plunger draws the drug fluid 154 from the refill container 150, whose collapsible pouch is exposed to the atmospheric pressure, into the dispensing chamber 2. In the meantime, no drug fluid would be moved from the reservoir chamber into the dispensing chamber as such movement would create vacuum pressure inside the reservoir chamber or inside the second compartment that prevents internal separation of the drug fluid or the filler fluid. Then a subsequent forward movement pushes the refill fluid from the dispensing chamber into the reservoir drug chamber 22 through the one-way valve opening 78. Due to high flow resistance in the needle or the use of a one-way valve inside the refill contain, the forward motion of the plunger does not push the drug fluid back into the refill container. A series of reciprocating motion of the plunger draws in the refill fluid from the refill container and delivers it into the reservoir drug chamber until both the dispensing and the reservoir drug chambers are full of the refill drug fluid.

Longer strokes of the forward and backward movements of the plunger can shorten the filling time required for total filling of the dispensing and the reservoir chambers. Simultaneously, during the refilling process the filler fluid 54 is returned to the filler fluid chamber through the flow gaps 58, which are in communication with the filler fluid inside the first and the second compartments. During these fluid movements the catheter flow channel 90 (shown in FIG. 3b) remains closed by the contact of the refill container needle against the catheter base walls. The refill container is of a passive type not using an externally-actuated plunger, which is a safety feature for avoiding any accidental injection. At the completion of a refilling process, at which both the dispensing and the reservoir chambers being full, the plunger and the second base are at their home positions with the two compressible chambers at their fully expanded shapes.

### Soft-Layer Filler-Fluid Chamber

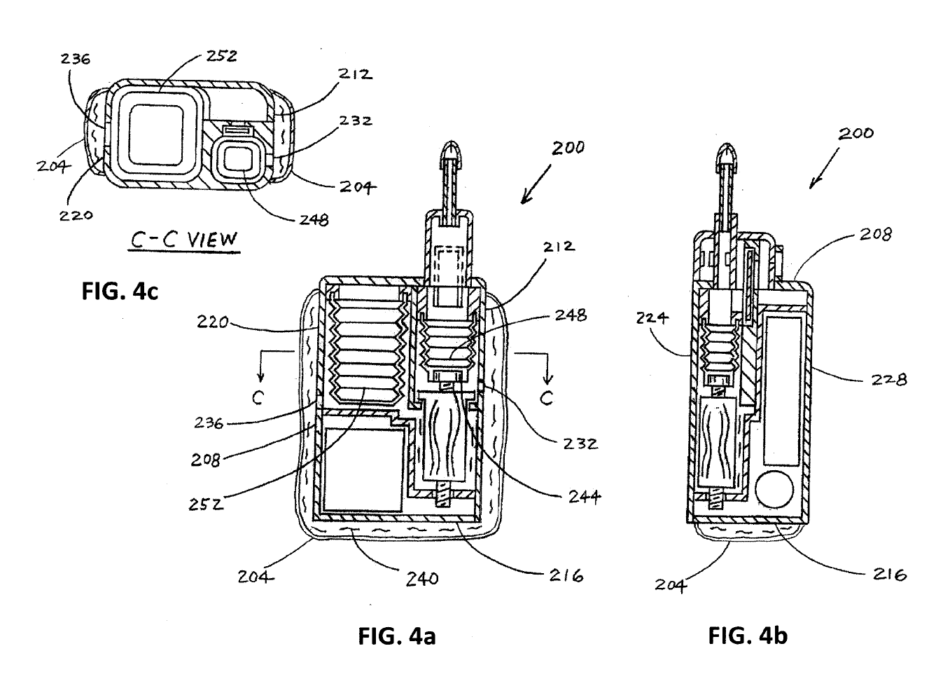

Instead of using a bellow configuration as shown in FIG. 1a, optionally a compressible filler fluid chamber may use collapsible soft layers for its chamber walls as shown in FIGS. 4a, 4b and 4c. FIG. 4a shows the soft layer 204 of the filler-fluid chamber attached externally to the housing walls 208 of the implantable drug delivery device 200. Preferably the soft layer is wrapped from first sidewall 212, around bottom wall 216, to second sidewall 220 of the housing walls 208. Other sidewalls 224, 228 are not attached with the soft layer for ease of manufacturing and manual handling prior to implantation procedures. FIGS. 4b and 4c show filler-fluid openings 232 and 236. First filler-fluid opening 232 on the first compartment wall 212 is for the entrance and exit of the filler fluid 240 behind the plunger 244 around the dispensing chamber 248 as the plunger moves forward and backward, respectively. On the other hand, the second filler-fluid opening 236 on the second compartment sidewall 220 is for the entrance and exit of the filler fluid around the reservoir chamber 252.

### Piezoelectric Motor

A drive means of an implantable drug delivery pump device of the present invention can be a piezoelectric motor, a stepper motor or an induction coil generating magnetic flux imparting motion on a magnetized plunger. A preferred embodiment is using a piezoelectric motor comprising threaded rod 112 driven by motor 20 as illustrated in FIG. 1a. The rotation of the threaded rod 112 causes forward and backward movements of plunger 18 corresponding to the rotational direction of the motor. Commercially a piezoelectric motor with the trade name “squiggle motor” is available to reduce the size of an implantable device. The assembly contains means for subjecting the threaded nut to ultrasonic vibration thereby causing the threaded shaft to simultaneously rotate and translate in the axial direction. A detailed description of a piezoelectric motor is given in U.S. Pat. No. 6,940,209 by Henderson.

Although the invention has been described with reference to particular embodiments, the description is only an example of the invention's application and should not be taken as a limitation. Various other adaptations and combinations of features of the embodiments disclosed are within the scope of the invention. For example, the compressible drug chamber as described in the bellows configuration can be used in a drug delivery device having only one drug chamber, which only uses a dispensing drug chamber without having a separate reservoir drug chamber. The present invention is for implantable drug delivery devices including implant and non-implant drug delivery devices. A stepper motor may be used as a drive means instead of a piezoelectric motor as described in the present invention. Also, an external power source and external controller may be used to reduce the size of an implantable pump device of the present invention. In such case the pump device needs to include an antenna and a RF receiver. Alternatively, a smaller size may be achieved by separating IC board and battery from the pump mechanism and implanted at different location away from the basic pump mechanism.

1. A drug delivery device comprising:
housing walls;
a compressible dispensing chamber containing a drug fluid having t first outlet and an enclosed first base, said compressible dispensing chamber being situated inside a first compartment supported by the housing walls with the first base attached to a plunger; and
a driver for imparting forward and backward movements of the plunger, said forward movement for moving the drug fluid toward the first outlet.

2. The drug delivery device of claim 1 further comprising:
a compressible reservoir chamber containing the drug fluid having a second outlet and an enclosed second base, said compressible reservoir chamber being situated inside a second compartment supported by the housing walls; and
a one-way valve positioned at the second outlet, said one-way valve closes when the plunger moves toward the first outlet and the one-way valve opens when the plunger moves away from the first outlet causing the drug fluid to flow from the reservoir chamber into the dispensing chamber.

3. The drug delivery device of claim 1 further comprising:
a compressible filler fluid chamber having collapsible walls containing an inert filler fluid, said compressible filler fluid chamber being attached to the housing walls and having flow paths in communication with the first compartment with said collapsible walls contracting as the filler fluid moves into the first compartment in response to the reduced volume of the drug fluid as said actuator moves forward to dispense the drug fluid out of the first outlet.

4. The drug delivery device of claim 2 further comprising:
a compressible filler fluid chamber having collapsible walls containing an inert filler fluid, said compressible filler chamber being attached to the housing walls and having flow paths in communication with the first and the second compartments with its collapsible walls contracting as the filler fluid moves into the first and the second compartments in response to the reduced volume of the drug fluid as said actuator moves forward to dispense the drug fluid cut of the first outlet.

5. A refill system of an implantable drug delivery device comprising:
an implantable drug delivery device having a compressible dispensing chamber containing a drug fluid having a first outlet and an enclosed first base, said first base being attached with a plunger and the first outlet having collapsible wails forming a outlet flow channel;
a refill container having a compressible drug chamber containing the drug fluid and a needle with an injection opening; and
a drive means for imparting forward and backward movements of the plunger, said forward movement for moving the drug fluid toward the first outlet and said backward movement draws the drug fluid from the refill container into the dispensing chamber when the needle is inserted into the first outlet to pinch on said collapsible walls to block the outlet flow channel.

6. The drug delivery device of claim 1, wherein said first outlet is attached with a catheter having a dispensing cap, said dispensing cap opens when the plunger moves toward the first outlet and closes when the plunger moves away from the first outlet.

7. The drug delivery devices of claim 6 wherein said first one-way valve being a slit valve.

8. The refill system of an implantable drug delivery device of claim 5, wherein said collapsible wails in the outlet flow channel are being deflected to activate an electrical switch by the insertion of the needle of said refill container.

9. The refill system of an implantable drug delivery device of claim 8, wherein said electrical switch is a contact switch having opposing electrode plates not in contact with the drug fluid with one electrode plate being attached to a backside of said collapsible walls of said outlet flow channel.

10. The refill system of an implantable drug delivery device of claim 5, wherein said drive means is a threaded rod and a motor to impart the rotation of the threaded rod to cause forward and backward movements of the plunger in the axial direction of the threaded rod corresponding to the rotational direction of the motor.

11. The drug delivery device of claim 1, wherein said driver is a piezoelectric motor comprising a threaded rod and piezoelectric plates, said threaded rod rotates when said piezoelectric plates being in piezoelectric vibration.

12. The drug delivery devices of claim 1, wherein said compressible dispensing chamber is of a bellows configuration.

13. The drug delivery device of claim 2, wherein said compressible reservoir chamber is of a bellows configuration.

14. The refill system of an implantable drug delivery device of claim 5, wherein said compressible dispensing chamber is of a bellows configuration.

15. The refill system of an implantable drug delivery device of claim 8, wherein said electrical switch is a Hall-effect switch.
